(19) United States
(12) Patent Application Publication
(10) Pub. No.: US 2012/0058534 A1
(43) Pub. Date: Mar. 8, 2012

(54) OPTIMIZED BIOGAS (BIOMETHANE) PRODUCTION PROCESS

(75) Inventors: Enos Loy Stover, Stillwater, OK (US); Ted Ross Stover, Stillwater, OK (US)

(73) Assignee: Stover & Associates, Inc.

(21) Appl. No.: 13/044,246

(22) Filed: Mar. 9, 2011

Related U.S. Application Data
(60) Provisional application No. 61/312,099, filed on Mar. 9, 2010.

(51) Int. Cl. C12P 5/02; D21C 7/12; D21C 1/00

(52) U.S. Cl. 435/167; 162/49; 162/238

(57) ABSTRACT

The present invention relates to a process for pretreatment of a feedstock in a pretreatment tank. Various parameters, such as oxidation-reduction potential, pH, and temperature, are monitored in the pretreatment tank to determine whether the oxidation-reduction potential, pH, and temperature are each within a predetermined range. The volume of feedstock inside the pretreatment tank is adjusted in response to a determination that one of the oxidation-reduction potential, pH, and temperature of the treated material are outside the corresponding predetermined ranges to maintain the oxidation-reduction potential, pH, and temperature of the treated material within operating conditions.

### CROSS REFERENCE TO RELATED APPLICATIONS

The present application claims the benefit under 35 U.S.C. 119(e) of U.S. Provisional Application Ser. No. 61/312,099, filed Mar. 9, 2010, which is hereby expressly incorporated by reference herein in its entirety.

### STATEMENT REGARDING FEDERALLY SPONSORED RESEARCH OR DEVELOPMENT

Not applicable.

### FIELD OF THE INVENTION

The present invention relates generally to treatment process schemes for processing of waste materials and various energy feedstocks (substrates), more particularly, but not by way of limitation, to primary treatment, preliminary treatment, and secondary anaerobic digestion treatment processes for wastes and energy feedstocks (substrates) in various forms to produce biogas (biomethane).

### BACKGROUND OF THE INVENTION

Various waste and feedstock materials that can be used for anaerobic digestion substrates have significantly different physical, chemical, and biochemical treatment characteristics. Some of these feedstock materials will require primary treatment for non-biodegradable solids removal, such as screening, sand and/or grit removal, gravity clarification, etc. Other feedstock substrates will require preliminary pretreatment to reduce the particle size, separate and/or release cellulose; hemicelluloses and/or lignins in order to increase the rates of hydrolysis and fermentation reactions required prior to anaerobic digestion conversion to biomethane. Examples of biomass feedstock are sugarcane, sugarcane extract, sugar beets, corn kernels, corn starch, paper, paper products, paper waste, wood, particle board, sawdust, agricultural waste, sewage, silage, grasses, rice hulls, bagasse, cotton, jute, hemp, flax, bamboo, sisal, abaca, straw, corn cobs, corn stover, switchgrass, alfalfa, hay, rice hulls, coconut hair, cotton, synthetic celluloses, seaweed, algae, and the like and/or mixtures thereof. Mechanical pretreatment for particle size reduction includes wet milling, dry milling, high pressure and/or high steam pretreatment, etc. Chemical pretreatment can include alkaline and acid hydrolysis, as well as hydrogen peroxide use under acidic conditions (Fentons Reagent type oxidation processes).

Certain feedstock substrates, such as materials containing lignocelluloses have to be hydrolyzed and enzymatically broken down to fermentable sugars prior to anaerobic digestion. The fermentable sugars then have to be fermented to volatile fatty acids (VFAs), such as acetic acid, prior to conversion to biomethane in the anaerobic digestion process. The pretreatment hydrolysis and production of fermentable sugars can be accomplished in a separate step prior to the anaerobic digestion process by the use of enzymes.

The viability of cellulosic biogas (biomethane) production depends on the sustainable bioenergy potential of lignocellulosic feedstocks through efficient anaerobic conversion processes to increase the efficiency of the biobased fuel production process. A biogas (biomethane) biobased economy should be technically feasible and cost-effective to provide value to the agricultural producers, manufacturers, and consumers. The biogas (biomethane) biobased fuel production facilities consist of different technologies and feedstocks and combinations of feedstocks appropriate to specific geographical regions. Diversification of feedstocks creates improved logistics and opportunities for energy production systems for potential use in agricultural based energy production. Enhancement and optimization of biogas production from lignocellulosic feedstocks is important for economic success of agricultural based bioenergy production.

The various feedstocks, with or without primary treatment, with or without preliminary treatment, are then blended in a preliminary treatment step for hydrolysis and preacidification (fermentation) to produce VFAs prior to the anaerobic digestion process. This blending preacidification step is first stage of treatment for single substrate components or co-digestion of multiple feedstock substrates. This blend preacidification step accomplishes the following:
Blends of feedstock substrates
Initiates hydrolysis of particulate substrates
Initiates fermentation to VFA production
Initiates conversion of organic nitrogen to ammonia-nitrogen
Creates the precursors for generated alkalinity production in the anaerobic digestion process

Pretreated feedstocks may be utilized in an anaerobic digestion process to produce biogas. During anaerobic digestion, the feedstock substrate(s), including the pretreated fermentation byproducts, are converted to methane, carbon dioxide, and hydrogen sulfide.

To this end, although treatment processes for processing waste and energy feedstocks is known in the art, further improvements are desirable to enhance the treatment of such feed material prior to anaerobic digestion and the production of biogas. It is to such a process that the present invention is directed.

### SUMMARY OF THE INVENTION

The present invention is a process for pretreatment of a feedstock. The feedstock is introduced into a pretreatment tank to form a bulk liquid. Pretreatment is performed of the feedstock in the pretreatment tank. Oxidation-reduction potential, pH, and temperature of the bulk liquid is monitored during pretreatment to determine whether the oxidation-reduction potential, pH, and temperature are each within a predetermined range. The volume of the feedstock in the pretreatment tank is adjusted in response to a determination that one of the oxidation-reduction potential, pH, or temperature of the bulk liquid is outside the corresponding predetermined ranges to maintain the oxidation-reduction potential, pH, and temperature of the bulk liquid within the predetermined ranges. Certain pretreatment processes reduce the particle size of the feedstock. The feedstock or combinations of various feedstocks may be blended to form a blended feed. The pretreatment of the feedstock or blended feed is preacidified. The pH of the pretreatment is within the range of from about 4.0 to about 9.5. The volatile fatty acids are between about 500 and about 5,000. When the process is performed mesophilic, the temperature is between about 80° F. and about 110° F. When the process is performed thermophilic, the temperature is between about 120° F. and about 160° F. The oxidation-reduction potential of the bulk liquid is maintained between about −150 mV and about −300 mV. The pH of the bulk liquid is maintained within a range of from about 4.0 to about 9.5. Various parameters may be monitored such as VFAs, COD, TKN, TP, TS, VS, TSS and VSS. Anaerobic digestion may be performed on the bulk liquid and a biogas having methane is produced. The carbon dioxide content of the biogas may be monitored and the carbon dioxide content of the biogas is from about 15% to about 40%. The pH is about 6.5 to about 8.0. The total alkalinity is from about 1,000 to about 10,000. The bicarbonate alkalinity is from about 500 to about 8,000.

The present invention is an apparatus for pretreatment of a feedstock. The apparatus includes a tank, a feed conduit, an effluent conduit, a temperature sensor, a pH sensor, and an oxidation-reduction potential sensor. The tank contains a bulk liquid. The feed conduit is operably coupled to the tank which facilitates introduction of feed material into the tank. The effluent conduit facilitates removal of treated effluent from the tank to an anaerobic digester. The temperature sensor measures the temperature of the bulk liquid. The pH sensor measures the pH of the bulk liquid. The oxidation-reduction potential sensor measures the oxidation-reduction potential of the bulk liquid. A microprocessor is connected to the oxidation-reduction potential, pH, and temperature sensors. The microprocessor automatically adjusts the level and volume of the bulk liquid in the tank in response to predetermined ranges of parameters such as oxidation-reduction potential, pH, temperature, VFAs, COD, TKN, TP, TS, VS, TSS, and VSS.

### BRIEF DESCRIPTION OF THE SEVERAL VIEWS OF THE DRAWINGS

FIG. 1 is a schematic diagram of one embodiment of a vessel constructed in accordance with the present invention utilized for practicing the process of the present invention.

FIG. 2 is a flow chart for a pretreatment control process.

### DETAILED DESCRIPTION OF THE INVENTION

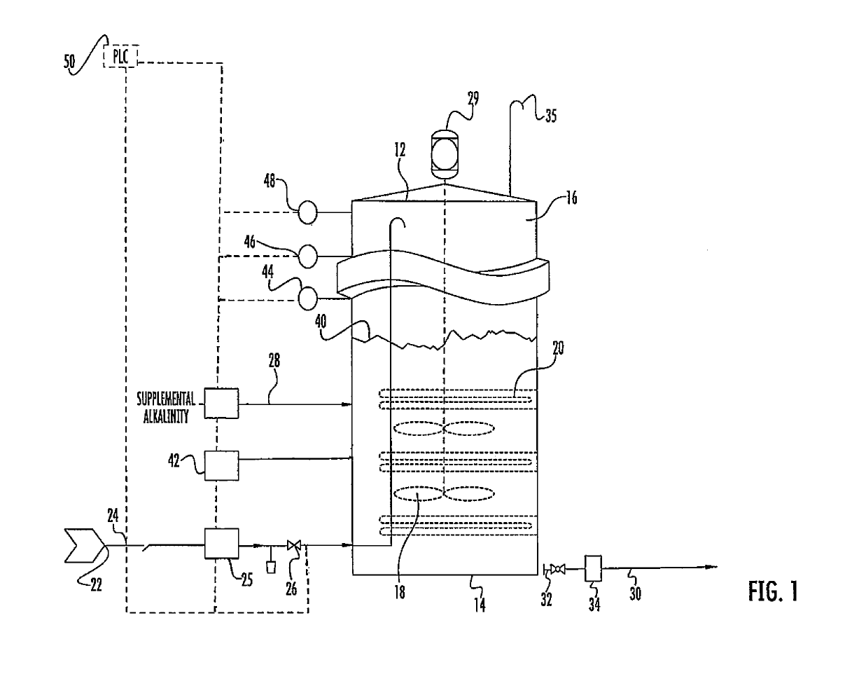

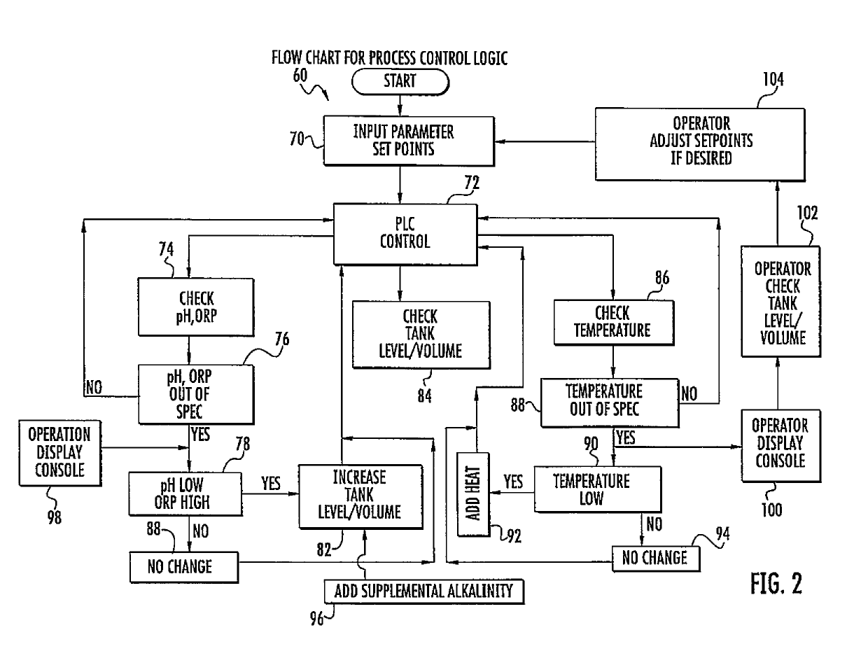

Referring now to the drawings, and more particularly to FIGS. 1 and 2, the present invention provides an improved process and apparatus for pretreatment of a feed material. The present invention provides an improved and enhanced biogas (biomethane) production process for conversion of various wastes, feedstocks, substrates, blends, or combinations of various feedstocks, etc. (e.g., biomass, materials containing lignocellulose, materials having inert non-biodegradable solids, etc.) individually or combined (co-digestion) through optimized design and operation of primary treatment, preliminary treatment, and secondary anaerobic digestion processes. It should be understood by one of ordinary skill in the art that the feedstock may be any material capable of undergoing treatment for producing biogas in accordance with the present invention as described herein. Further, although the present invention discusses using the pretreatment process in conjunction with anaerobic digestion, by way of example, it should be understood by one of ordinary skill in the art that the present invention may be used for the pretreatment of feed material for aerobic and anaerobic digestion processes.

Shown therein is a tank 10 constructed in accordance with the present invention for pretreatment of the feed material. The tank 10, in one embodiment of the present invention as described herein, is a variable level tank, wherein a liquid level in the tank is constantly changing. However, it should be understood by one of ordinary skill in the art that any tank may be utilized for a pretreatment process so long as the tank functions in accordance with the present invention. Further, it should be understood by one of ordinary skill in the art that although a single tank 10 is shown as described herein, a plurality of tanks may be utilized so long as the plurality of tanks functions in accordance with the present invention as described herein.

The tank 10 includes a vessel 12 having a bottom 14, a top 16, and at least one agitator 18 for mixing the feed material. The tank 10 may further be provided with a heating apparatus 20, such as heat coils, for heating the feed material (substrates) in the tank 10. The heating apparatus 20 is utilized in the event the heating requirements (mesophilic or thermophilic) are not satisfied by a fuel value content or the temperature of the feed material to be treated. Such a supplemental heating system allows the tank bulk liquid temperature to be maintained within the desired temperature range.

A feed source(s) 22, designates the feed material to be treated by the pretreatment process, which may be any soluble, slurry, or solid waste, or various combinations thereof, having organic and/or other constituents particularly suitable for a pretreatment process as further described herein.

The feed material is pumped to the vessel 12 by a feed pumping system (not shown) by conduit 24 for ultimate mixing with the tank contents (or “bulk liquid”) of tank 10. To regulate the flow of feed material to the tank 10, a flow meter 25 and a control valve 26 are provided in the conduit 24. Alternatively, the flow of influent feed material can be regulated by using a variable speed pump (not shown). A conduit 28 is provided for adding supplemental alkalinity as described hereinafter.

Upon entering the tank 10, the feed material is mixed by the at least one agitator 18 according to one of the treatment processes as described herein. A motor 29 controls the speed and direction of the at least one agitator 18. In the variable volume (or variable liquid level) mode, the treated effluent is removed from the tank vessel 12 through a lower effluent 30. Effluent 30 of the tank 10 is removed and sent to an anaerobic digester(s) (not shown) for the production of biogas. The effluent 30 is monitored and regulated by an effluent flow meter 32 and effluent control valve 34. A vent 35 is provided at the top 16 of the tank 10 for venting air and gases out of the tank 10.

Control of a liquid line level 40 is necessary to ensure optimal performance of the tank 10. To control the volume/liquid level 40, the tank 10 is equipped with an overflow line which serves to set the maximum height of the liquid in the tank. The tank 10 is provided with a liquid level sensor 42 for monitoring the level of the liquid in the tank 10.

The improved pretreatment process utilizes specific parameter monitoring to optimize and control the pretreatment process. The parameters are monitored in the tank 10 bulk liquid produced. Supplemental alkalinity and specific formulations of biological growth micronutrients are utilized for further enhancing and optimizing of the pretreatment process.

In accordance with the present invention, optimization of the pretreatment process is achieved by on-line monitoring and controlling the temperature, pH, and oxidation-reduction potential (ORP) of the tank 10 bulk liquid to maintain these critical parameters within appropriate ranges. The tank 10 is provided with a temperature sensor 44, a pH sensor 46, and an ORP sensor 48 to monitor these parameters of the bulk liquid. The volume of the feedstock in the tank 10 is adjusted in response to a determination that one of the ORP, pH, or temperature of the bulk liquid (pretreatment) is outside the corresponding predetermined range to maintain the ORP, pH, and temperature of the bulk liquid within the predetermined ranges.

Different feedstocks have different physical and chemical characteristics and require different primary and preliminary treatment steps (processes) prior to the anaerobic digestion step (process) in order to enhance and optimize biogas (biomethane) production. For example, feedstocks containing inert non-biodegradable solids may require primary treatment for inert solids removal. Industrial wastes typically require screening to remove large solids and trash that get discharged into the process sewer lines. Other feedstocks may require inert solids removal. For example, hen laying manure which contains large amounts of grit which is removed prior to anaerobic digestion. Sand and/or grit removal can be accomplished by various grit removal processes, such as gravity clarification, hydrocyclones, etc. The non-biodegradable inert solids should be removed to minimize wear and tear on pumps, particle sizing equipment, etc., as well as to prevent inert solids build-up and accumulation in a tank used for anaerobic digestion, reducing the effective tank working volume.

If lignocellulosic feedstocks are utilized, primary pretreatment is conducted for particle size reduction, separation and release of cellulose, hemicelluloses, and lignins, in order to increase the rates of biological hydrolysis and fermentation reactions required prior to anaerobic conversion to biogas (biomethane). Particle size reduction can be accomplished by mechanical means such as milling and/or use of high pressure and steam or chemical means such as alkaline or acid hydrolysis reactions. It should be understood by one of ordinary skill in the art that any process used for reducing particle size of a lignocellulosic feedstock can be utilized so long as the process functions in accordance with the present invention as described herein.

After primary treatment of lignocellulose feedstocks, the substrates are hydrolyzed and enzymatically broken down to fermentable sugars as precursors to anaerobic digestion. The fermentable sugars are then converted to VFAs, such as acetic acid, prior to conversion to methane. The enzymatic hydrolysis and fermentation process may be initiated in a preliminary pretreatment step prior to anaerobic digestion in order to enhance and optimize the methane process during anaerobic digestion. However, it should be understood by one of ordinary skill in the art that hydrolysis and preliminary fermentation reactions may also occur during anaerobic digestion.

A number of selected enzymes, such as various hydrolase type enzymes, including cellulases, glucosidases, xylanases, glucanases, hemicellulases, proteases and amino acid oxidases, lipases, etc., can be used in the pretreatment step under controlled environmental conditions (pH, temperature, etc.) to enhance and optimize hydrolysis and fermentation prior to anaerobic digestion. Hydrolysis can be performed under mesophilic (about 80° F. to about 110° F.) or thermophilic (about 120° F. to 160° F.) conditions. The lignocellulosic containing feedstocks are hydrolyzed to glucose and other sugars which are the precursors for producing the fermentation products, such as acetic acid. Hydrolysis can be performed in a continuous, semi-continuous, or batch fed process. Fermentation can be carried out in the same step, as the enzymatic hydrolysis, but preferably, is performed in a separate blend preacidification step, that can also be operated under mesophilic or thermophilic conditions.

Another preliminary pretreatment step is blending and preacidification. The feedstock and/or various feedstocks are blended in a reaction vessel 12 for comingling the substrates, initiating or continuing the hydrolysis (depending on the previous pretreatment steps employed which are feedstock specific), and performing the fermentation reactions. The fermentation reactions convert the feedstock components (carbohydrates, proteins, and lipids) into VFAs, such as acetic acid. The VFAs are the precursors to methane production in the next step, anaerobic digestion. The blend and preacidification process can be performed either under mesophilic or thermophilic conditions.

The blending and preacidification step serves other important functions in addition to the hydrolysis and fermentation reactions. One aspect of the fermentation of protein containing feedstocks is the release of organically bound nitrogen to ammonia-nitrogen (NH₃—N). Proteinaceous feedstocks generate excess nitrogen in the ammonia form which reacts with CO₂ in the fermentation tank bulk liquid and the anaerobic tank bulk liquid to produce ammonium bicarbonate alkalinity (NH₄HCO₃) (from about 500 to about 8,000). A significant portion of the CO₂ that is produced from the biological activity reacts with the ammonia and remains in the aqueous phase (bulk liquid). For each mg/L of NH₃—N formed, about 5.6 mg/L of NH₄HCO₃ alkalinity is formed, which is equivalent to about 3.6 mg/L of calcium carbonate (CaCO₃) alkalinity. The ammonium bicarbonate alkalinity causes the pretreatment fermentation tank and the anaerobic tank bulk liquid pH to increase; with highly proteinaceous wastes, the pH can increase into the 8.0 plus pH range.

The pH range of pretreatment for the present invention is within the range of about 4.0 to about 9.5. A pH range of about 4.0 to about 6.5 has been reported as optimal for the preacidification step. A pH range of about 6.5 to about 8.0 has been reported as optimal for anaerobic methane production. A phased approach of first step preacidification may be beneficial to the anaerobic process.

In the pretreatment step(s) (primary treatment, preliminary treatment, and/or secondary anaerobic treatment) of the feedstocks, prior to anaerobic digestion, the following parameters may be monitored and assessed: pH, temperature, ORP, total Kjeldahl nitrogen (TKN), total phosphorus (TP), total solids (TS), total volatile solids (VS), VFAs, chemical oxygen demand (COD), total suspended solids (TSS), volatile suspended solids (VSS), and alkalinity.

The various monitor and control elements of the pretreatment tank are regulated automatically by means of a programmable logic controller (PLC) 50, which includes a computer linked to the various monitoring and control elements. Various parameter setpoints are initially established by the operator. The parameter setpoints can include a desired temperature range within which the process operates and desired pH and ORP operating ranges for the process.

The parameter setpoints are provided to a microprocessor, such as the PLC 50, which proceeds to monitor the operation of the pretreatment process. More particularly, the temperature, pH, ORP, level/volume of the tank 10 are periodically measured and checked to determine whether these measured parameters are within the selected operating ranges. When the measured parameters remain within the selected ranges, no adjustments are made to the control elements. However, when the measured parameters fall outside the selected operating ranges, operational process control changes are required.

An example of one embodiment of such an arrangement is shown in FIG. 2. More particularly, FIG. 2 provides a flow chart for a pretreatment control process 60 in accordance with a preferred embodiment of the present invention. Each of the steps of the process will be discussed in turn.

Beginning at step 70, various parameter setpoints are initially established by the operator. As discussed above, such parameter setpoints can include a desired temperature range within which the pretreatment process (mesophilic—between about 80° F. and about 110° F.; thermophilic—between about 120° F. and about 160° F.) operates and a desired ORP, pH, and level/volume of the tank range for the process. It will be understood that the desired ORP range will typically be between about −150 mV and about −300 mV. The pH range is between about 4.0 and about 9.5.

At step 72, the parameter setpoints are provided to a microprocessor of the PLC 50, which proceeds to monitor the operation of the pretreatment process. More particularly, as indicated at step 74, the pH and/or ORP are periodically measured and checked to determine whether the measured pH and ORP are within the selected pH and ORP ranges. When the measured pH and ORP remain within the selected ranges, as shown by decision step 76, no adjustments are made to the control elements.

However, when the measured pH and ORP fall outside the selected pH and ORP ranges, the flow continues from decision step 76 to decision step 78, which determines whether the out of spec pH and/or ORP are outside the established ranges. If not, the flow continues to step 80 where there is no change to the level/volume in the tank 10. On the other hand, if the out of spec pH and/or ORP change to where the pH is low and the ORP is high, the flow continues to step 82 where the microprocessor operates to increase the level/volume of the tank 10 by the setpoint value increment selected at step 72. Preferably, the microprocessor initiates an internal timer upon detection of an out of spec pH and/or ORP and does not proceed to adjust the rate of feed material into the tank 10 until expiration of the timer. This prevents undesired adjustments to spurious pH and/or ORP readings.

Additionally, as indicated at step 84, the level/volume of the liquid in the tank is also monitored.

Additionally, from step 72, simultaneously, the PLC 50 monitors other parameters of the process. As indicated at step 86, the temperature is periodically measured and checked to determine whether the measured temperature is within the selected temperature ranges. When the measured temperature remains within the selected ranges, as shown by decision step 88, no adjustments are made to control elements.

However, when the measured temperature falls outside the selected temperature range, the flow continues from decision step 88 to decision step 90, which determines whether the temperature is outside the established range. If so, the flow continues to step 92 where the microprocessor operates to add heat into the tank 10 by the setpoint value increment selected at step 72. On the other hand, if the out of spec temperature is within the established range, the flow continues to step 94 where there is no change. Preferably the microprocessor initiates an internal timer upon detection of an out of spec temperature and does not proceed to adjust the level/volume of liquid in the tank until expiration of the timer. This prevents undesired adjustments to spurious temperature readings.

In step 96, supplemental alkalinity may be added to the tank 10, if decided by step 72.

Continuing with the flow of FIG. 2, at such time that the measured parameters are determined to be out of spec, the process also continues from the decision step 76 to step 98 and from decision step 88 to step 100, where an indication is preferably made on an operator display console to inform the operator that the measured parameters are out of spec. This allows the operator to perform a manual check of the liquid level/volume in the tank 10, as shown at step 102, and to make any changes to the parameter setpoints at step 104.

The following is the reaction chemistry of acetic acid and acetate salts which is important to understanding the chemical and biochemical reactions that occur in both the fermentation and anaerobic systems as a function of these relative chemicals and their reaction products of metabolism:

Acetate Forms of Concern:

| | |
|---|---|
| Acetic acid | CH₃COOH @ low pH |
| Sodium acetate | Na(CH₃COO) |
| Potassium acetate | K(CH₃COO) |
| Calcium acetate | Ca(CH₃COO)₂ |

Acetic Acid Reaction Chemistry:

CH₃COOH→CH₄+CO₂

CO₂+4H₂→CH₄+2H₂O

Acetic acid forms methane and carbon dioxide in an anaerobic digester. The CO₂ reacts to produce both CH₄ and carbonic acid which represents the major acidity produced by anaerobic treatment, as follows:

At pH 8.4, the carbonate ion is converted to bicarbonate ion, as follows:

CO₃²⁻+H⁺→HCO₃⁻

Below pH 8.3, the carbonate ion is converted to carbonic acid, as follows:

HCO₃⁻+H⁺→H₂CO₃

One requirement for alkalinity in anaerobic systems is neutralization of the high H₂CO₃ which results from the high partial pressure of CO₂ in the system. The alkalinity requirement for VFA neutralization is small compared to that for H₂CO₃.

The chemical/biochemical reactions are the normal process for anaerobic treatment. The acetic acid metabolism and associated acidity/alkalinity relationships associated with the naturally occurring carbonate/bicarbonate relationships normally govern the process. However, if the acetic acid exists as acetate salts during the preacidification step, the reactions can change.

Sodium and potassium acetate will react the same, as follows:

Na(CH₃COO)+H₂O→CH₄+NaHCO₃

K(CH₃COO)+H₂O→CH₄+KHCO₃

NaHCO₃ and KHCO₃ are alkalinity. NaHCO₃ produces high alkalinity and buffering capacity to keep the pH high. Also, there is no CO₂ produced. Therefore, there can be no carbonic acid produced. Both sodium and potassium acetate produce excessive alkalinity (generated alkalinity) and high pH caused by lack of CO₂ production.

Calcium acetate reacts as follows:

Ca(CH₃COO)₂+H₂O→2CH₄+CO₂+CaCO₃

CO₂ is produced which prevents an excessive increase in the system alkalinity and pH. The divalent cation magnesium reacts in the same manner as calcium.

When preacidification is employed for high strength wastes prior to anaerobic digestion, the amount of acetate salts and/or potassium salts fed to the process are monitored and controlled to improve performance of the process. The sodium and potassium bicarbonate alkalinity generated can be excessive which can be observed both by increased alkalinity production in the bulk liquid and decreased CO₂ content in the biogas.

The salts of acetic acid show up as alkalinity in an alkalinity test, but are not available for neutralization of additional VFAs, even though they may constitute a major fraction of the total alkalinity. Therefore, a distinction between bicarbonate alkalinity and the total alkalinity (which includes the salts of VFAs) becomes important when high concentrations of acetic acid salts are present. Bicarbonate alkalinity can be calculated as follows:

B-Alk=Total Alk−[(0.83)(0.85)(VFA)]

Any cation except H⁺ keeps CH₃COO⁻ in the alkalinity form.

The following parameters are monitored and controlled:
Preacidification VFAs (VFA ranges depend on types of substrates/feed material and type of digestion process) (about 500 to about 5,000);
Anaerobic biogas CO₂ content (about 15% to about 40%);
Anaerobic effluent pH (about 4.0 to about 9.5) and alkalinity; and
Total alkalinity (about 1,000 to about 10,000) and bicarbonate alkalinity (about 500 to about 8,000) relationships.

Monitoring the parameters is important for optimized biogas (biomethane) production. If the parameters begin falling outside of target operational ranges, it will be important to make operational adjustments to prevent process upsets and possible anaerobic system failure. The various monitor and control elements of the pretreatment processes are regulated automatically by means of the PLC, which includes a computer linked to the various monitoring and control elements.

Various parameter setpoints are initially established by an operator. The parameter setpoints can include a desired temperature range within which the process operates and desired pH and ORP operating ranges for the process.

The parameter setpoints are provided to the microprocessor which proceeds to monitor the operation of the pretreatment processes. More particularly, the temperature, pH, and ORP are periodically measured and checked to determine whether these measured parameters are within the selected operating ranges. When the measured parameters remain within the selected ranges, no adjustments are made to the control elements. However, when the measured parameters fall outside the selected operating ranges, operational process control changes are required.

In accordance with the present process, VFAs are typically monitored using wet chemistry techniques. The range of VFAs in the present invention are between about 500 and about 5,000. Alkalinity is also typically monitored with wet chemistry techniques, and both VFAs and alkalinity are used in the process control algorithms with PLC process controls. Input parameters include VFAs and alkalinity for tank 10 biological activity and health; along with COD and VS (measures of substrate/feedstock strength or biomethane generation potential).

Generated alkalinity in the bulk liquid of the anaerobic digestion tank can have impacts on decreasing the CO₂ content of the biogas, as well as maintaining pH and alkalinity control in the anaerobic digestion bulk liquid. The CO₂ content of the biogas is from about 15% to about 40%. Ammonia-nitrogen reacts with CO₂ to produce ammonium bicarbonate alkalinity in the bulk liquid which generates alkalinity assisting with pH control while at the same time reducing the amount of CO₂ emitted in the biogas. Therefore, the formation of ammonium bicarbonate reduces the CO₂ content of the biogas while having no effect on the H₂S content of the biogas. The monovalent cations, sodium and potassium, under proper pretreatment preacidification conditions produce VFA salts of sodium and potassium bicarbonate to help with pH and alkalinity control in the anaerobic tank bulk liquid. The reactions have no effect on the bulk liquid solubility of CO₂ and H₂S, however, the generated alkalinity consumes CO₂, thus reducing the amount of CO₂ emitted in the biogas.

From the above description, it is clear that the present invention is well adapted to carry out the objects and to attain the advantages mentioned herein, as well as those inherent in the invention. While presently preferred embodiments of the invention have been described for purposes of this disclosure, it will be understood that numerous changes may be made which will readily suggest themselves to those skilled in the art and which are accomplished within the spirit of the invention as disclosed and claimed herein.

What is claimed is:

1. A process for pretreatment of a feedstock, comprising the steps of:
introducing the feedstock into a pretreatment tank to form a bulk liquid;
performing pretreatment of the feedstock in the pretreatment tank;
monitoring oxidation-reduction potential, pH, and temperature of the bulk liquid during pretreatment to determine whether the oxidation-reduction potential, pH, and temperature are each within a predetermined range; and
adjusting the volume of the feedstock in the pretreatment tank in response to a determination that one of the oxidation-reduction potential, pH, or temperature of the bulk liquid is outside the corresponding predetermined ranges to maintain the oxidation-reduction potential, pH, and temperature of the bulk liquid within the predetermined ranges.

2. The process of claim 1, wherein the pretreatment reduces the particle size of the feedstock.

3. The process of claim 1, further comprising the step of:
blending the feedstock or combinations of various feedstocks to form a blended feed.

4. The process of claim 3, wherein the pretreatment of the feedstock or blended feed is preacidified.

5. The process of claim 4, wherein the pH of the pretreatment is within the range of from about 4.0 to about 9.5.

6. The process of claim 4, wherein the volatile fatty acids are between about 500 and about 5,000.

7. The process of claim 1, wherein the process is performed mesophilic at temperatures between about 80° F. and about 110° F.

8. The process of claim 1, wherein the process is performed thermophilic at temperatures between about 120° F. and about 160° F.

9. The process of claim 1, further comprising a step of:
maintaining the oxidation-reduction potential of the treated material between about −150 mV and about −300 mV.

10. The process of claim 1, wherein the process is performed mesophilic at temperatures between about 80° F. and about 110° F.

11. The process of claim 1, wherein the process is performed thermophilic at temperatures between about 120° F. and about 160° F.

12. The process of claim 1, further comprising the step of:
maintaining the pH of the treated material within a range of from about 4.0 to about 9.5.

13. The process of claim 1, further comprising the step of:
monitoring parameters selected from the group consisting of VFAs, COD, TKN, TP, TS, VS, TSS and VSS.

14. The process of claim 1, further comprising the steps of:
performing anaerobic digestion on the treated material; and
producing a biogas having methane.

15. The process of claim 14, further comprising the step of:
monitoring the carbon dioxide content of the biogas.

16. The process of claim 15, wherein the carbon dioxide content of the biogas is from about 15% to about 40%.

17. The process of claim 14, wherein the pH in anaerobic digestion is from about 6.5 to about 8.0.

18. The process of claim 14, wherein the total alkalinity is from about 1,000 to about 10,000.

19. The process of claim 14, wherein the bicarbonate alkalinity is from about 500 to about 8,000.

20. An apparatus for pretreatment of a feedstock, comprising:
a tank containing a bulk liquid;
a feed conduit operably coupled to the tank which facilitates introduction of feed material into the tank;
an effluent conduit which facilitates removal of treated effluent from the tank to an anaerobic digester;
a temperature sensor for measuring the temperature of the bulk liquid;
a pH sensor for measuring the pH of the bulk liquid; and
an oxidation-reduction potential sensor for measuring the oxidation-reduction potential of the bulk liquid.

21. The apparatus of claim 20, further comprising:
a microprocessor connected to the oxidation-reduction potential, pH, and temperature sensors.

22. The apparatus of claim 21, wherein the microprocessor automatically adjusts the level and volume of the bulk liquid in the tank in response to predetermined ranges of parameters selected from the group consisting of oxidation-reduction potential, pH, or temperature.

23. The apparatus of claim 21, wherein the microprocessor automatically adjusts the level and volume of the bulk liquid in the tank in response to predetermined ranges of parameters selected from the group consisting of VFAs, COD, TKN, TP, TS, VS, TSS, or VSS.
